(19) United States
(12) Patent Application Publication
(10) Pub. No.: US 2014/0026905 A1
(43) Pub. Date: Jan. 30, 2014

(54) APPARATUS AND METHOD OF CONTROLLING THE RATE OF NICOTINE DELIVERY

(71) Applicant: Nuvo Research Inc., Mississauga (CA)

(72) Inventors: Jie Zhang, Salt Lake City, UT (US); Wade A. Hull, Kaysville, UT (US)

(21) Appl. No.: 13/787,543

(22) Filed: Mar. 6, 2013

Related U.S. Application Data
(60) Provisional application No. 61/676,522, filed on Jul. 27, 2012.

(51) Int. Cl. A61K 9/70; F24J 1/00

(52) U.S. Cl. CPC A61K 9/703; F24J 1/00
USPC 131/270; 126/263.01

(57) ABSTRACT

The present disclosure provides for an apparatus and related method for controlling the rate of nicotine delivery into systemic circulation of a subject. The method comprises transdermally administering nicotine at a topical administration site of a subject; achieving a steady state plasma concentration of nicotine in the subject; and activating a temperature modification apparatus over the topical administration site after achieving the steady state. The temperature modification apparatus can be configured to generate heat for a predetermined period of time. Further, about 5 to about 30 minutes following activation of the temperature modification apparatus, the subject's nicotine plasma concentration increases by at least about 5% over the subject's steady state nicotine plasma concentration.

This application claims the benefit of U.S. Provisional Patent Application No. 61/676,522, filed Jul. 27, 2012.

### BACKGROUND

Transdermal nicotine delivery systems, such as nicotine patches, are commonly used to aid people in their attempts to quit smoking. The patches help by reducing a subject's cravings for tobacco. For example, each patch contains a specific amount of nicotine embedded in a pad or gel which is transdermally delivered to the subject over a period of time. Typically, nicotine patches are designed to create constant lower levels (steady state) of nicotine in the body of the user. However, generally the levels of nicotine delivered by the patches are lower than levels that would result during the smoking of a cigarette or immediately thereafter. Thus, a cigarette provides a relatively sudden spike or jolt of nicotine in the user, but the nicotine delivered from a nicotine patch is typically relatively constant once the nicotine blood serum level reaches a steady state.

Although nicotine patches are effective aids for some people who are trying to quit smoking, others find that their cravings occasionally exceed the nicotine dose levels provided by the patch, which in turn can result in a failed attempt to quit smoking.

### BRIEF DESCRIPTION OF THE DRAWINGS

FIG. 1 is a schematic representation of an exemplary integrated system of the present disclosure. This example system includes a transdermal nicotine delivery patch attached to a transdermal temperature modification apparatus.

FIGS. 2A and 2B are graphs which plot the heating profile of exemplary exothermic temperature modification apparatuses of the present disclosure.

FIG. 3 is graph which plots the mean nicotine plasma concentrations following application of a 7mg nicotine transdermal patch and subsequent activation of a temperature modification apparatus (at t=6 hours).

### SUMMARY

The present disclosure provides for an apparatus and related method for controlling the rate of nicotine delivery into systemic circulation of a subject. With respect to the method, steps can include transdermally administering nicotine at a topical administration site of a subject; achieving a steady state plasma or a pre-activation plasma concentration of nicotine in the subject; and activating a temperature modification apparatus over the topical administration site after achieving the steady state concentration of nicotine or following a pre-set period of time. The temperature modification apparatus can be configured to generate heat for a predetermined period of time. Further, about 5 to about 30 minutes following activation of the temperature modification apparatus, the subject's nicotine plasma concentration increases by at least about 5% over the subject's steady state nicotine plasma concentration.

In another embodiment, a temperature modification apparatus for use with a transdermal nicotine system is provided. The apparatus can include an exothermic chemical composition layer, an air impermeable layer disposed on an upper surface of the chemical composition layer and having one or more holes therein, an activation tab, and an adhesive layer. The activation tab can be removably adhered to an upper surface of the air impermeable layer and can be configured to cover the one or more holes in the air impermeable layer to inhibit the passage of air through the holes prior to removal of the activation tab. The adhesive layer can be disposed on a lower surface of one or both of the exothermic chemical composition layer and the lower surface of the air impermeable layer, and can be configured to adhere the temperature modification apparatus to one or both of a skin surface and the transdermal nicotine delivery system.

### DETAILED DESCRIPTION

Before particular embodiments of the present invention are disclosed and described, it is to be understood that this disclosure is not limited to the particular process and materials disclosed herein as such may vary to some degree. It is also to be understood that the terminology used herein is used for the purpose of describing particular embodiments only and is not intended to be limiting, as the scope of the present disclosure will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a temperature modification apparatus” includes reference to one or more of such apparatus.

“Skin” is defined to include human skin (intact, diseased, ulcerous, or broken), and mucosal surfaces that are usually at least partially exposed to air such as lips, genital and anal mucosa, and nasal and oral mucosa.

As used herein, “steady state” refers to the concentration of nicotine in a body fluid (usually plasma) when the rates of drug administration and drug elimination are substantially equal. This can be measured in terms of ng/mL, for example. Thus, an “increase” in the nicotine in the body can be quantified in terms of a percentage increase in the plasma concentration (also ng/mL). As an example, if the steady state of nicotine in the blood serum is about 6 ng/mL, and then the plasma is rapidly increased to about 7 ng/mL, the increase in plasma concentration is about 17%.

As used herein, the term “pre-activation plasma concentration” refers to nicotine plasma concentration levels following the application of a transdermal nicotine delivery system for a pre-set period of time and prior to the activation of a temperature modification apparatus.

As used herein, the term “drug depot” refers to a region in a subject's skin or sub-skin tissue in which a drug or active agent (e.g. nicotine) has been collected before being gradually released into systemic circulation. Typically, when a transdermal drug delivery system is applied to skin, the drug begins to transport out of the formulation and into the skin. The drug then enters blood vessels and tissues under the skin, and is taken into the system circulation of the body by the blood. For many drugs, a significant portion of the dermally absorbed drug is stored in the skin and/or tissues under the skin (i.e. the drug depot) before being gradually taken into the systemic circulation. This can be likened to a “reservoir” of drug that collects under the skin, and is passed into systemic circulation from the depot and into systemic circulation. Thus, when the drug has reached a steady state in the body, a reservoir or depot of drug remains under the patch, and can be rapidly dumped into circulation by application of heat in accordance with examples of the present disclosure.

As used herein, the term “pre-set period of time” refers to a period of time following the application of a transdermal nicotine delivery system but before the activation of a temperature modification apparatus. In one embodiment, the pre-set period of time can be at least about 1 hour. In another embodiment, the pre-set period of time can be at least about 2 hours. Other pre-set periods of time can range from 1 to 24 hours, 1 to 12 hours, 2 to 12 hours, 2 to 10 hours, and 3 to 8 hours.

As used herein, a “temperature modification apparatus” is defined as an apparatus capable of providing controlled heating to a pre-determined narrow temperature range and for a predetermined duration. A temperature control apparatus that can be used in accordance with the methods of the present disclosure can be configured to generate heat promptly when activated. The duration and magnitude of the heating can be achieved through specific designs of the temperature modification apparatus. For example, when the temperature modification apparatus is an exothermic temperature modification apparatus, the magnitude and duration of the heating can be controlled based on factors such as ratios and make-up of the exothermic chemical compositions, physical constraints on the exothermic chemical reaction such as limitations on the air flow or oxygen contact with the exothermic material, special configuration of individual heating elements, conductivity of materials used, etc. The temperature control apparatus is not limited to the exothermic temperature control apparatuses, as any heating device that provides heating within the appropriate temperature ranges can be used in accordance with methods disclosed herein.

As used herein, “subject” refers to a human, male or female.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein. Furthermore, when using the term “about” in a range, it is understood that the range also includes the exact numerical values of the range. For example, the range “about 36° C. to about 42° C.” explicitly includes and provides an additional direct teaching of the range “36° C. to 42° C.”

As used herein, the term “substantially” or “substantial” refers to the complete or nearly complete extent or degree of an action, characteristic, property, state, structure, item, or result. For example, an object that is “substantially” over a given area would mean that the object is either completely over or nearly completely over that area. The exact allowable degree of deviation from absolute completeness may in some cases depend on the specific context. However, generally speaking, the nearness of completion will be so as to have the same overall result as if absolute and total completion were obtained.

As used herein, a plurality of items, such as compounds, and/or heating mechanisms, may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0 mm” should be interpreted to include not only the explicitly recited values of about 0.01 mm to about 2.0 mm, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

With this background in mind, the present disclosure is drawn to an apparatus and related method for controlling the rate of nicotine delivery into systemic circulation of a subject. The method can comprise transdermally administering nicotine at a topical administration site of a subject; achieving a steady state plasma concentration of nicotine in the subject; and activating a temperature modification apparatus over the topical administration site after achieving the steady state. The temperature modification apparatus can be configured to generate heat for a predetermined period of time. Further, about 5 to about 30 minutes following activation of the temperature modification apparatus, the subject's nicotine plasma concentration increases by at least about 5% over the subject's steady state nicotine plasma concentration. Optionally, the activation of the temperature modification device can be repeated once the steady state has been re-established.

The method of the present disclosure can be accomplished utilizing known and commercially available transdermal nicotine patch, plaster, peel, ointment, lotion, cream, or other topical treatment or methods for providing transdermal nicotine delivery. Non-limiting examples of commercially available transdermal nicotine delivery systems or patches that can be used include Habitrol®, Nicoderm®, Nicoderm CQ®, Nicotrol®, or any other currently and previously available transdermal nicotine device or system. The strength or amount of transdermal delivery system can be such that it delivers about 5 mg nicotine/24 hours to about 25 mg nicotine/24 hours. Common strength for commercially available transdermal nicotine patches include 7 mg nicotine/24 hours, 14 mg nicotine/24 hours, 15 mg nicotine/16 hours, and 21 mg nicotine/24 hours. The transdermal system used in the disclosed method can have a skin contact area where the nicotine is delivered from the system to a subject's skin surface. The size of the skin contact area can vary depending on various factors, including the concentration of nicotine in the patch. The skin contact area can have an area of 2 cm² to 100 cm², 7 cm² to 75 cm², 10 cm² to 50 cm², or 15 cm² to 35 cm². In various other embodiments, other size ranges may be appropriate. Other means for achieving transdermal delivery of nicotine can also be used, in particular if they are able to create an intradermal or subcutaneous depot of nicotine.

In another embodiment, a temperature modification apparatus, such as one that be used in the methods disclosed herein, is provided. The apparatus can include an exothermic chemical composition layer, an air impermeable layer disposed on an upper surface of the chemical composition layer and having one or more holes therein, an activation tab, and an adhesive layer. The activation tab can be removably adhered to an upper surface of the air impermeable layer and being configured to cover the one or more holes in the air impermeable layer and inhibit the passage of air into and through the holes prior to removal of the activation tab. The adhesive layer can be disposed on a lower surface of one or both of the exothermic chemical composition layer and the lower surface of the air impermeable layer and can be configured to adhere the temperature modification apparatus to one or both a skin surface and a transdermal nicotine delivery system.

The temperature modification apparatus of the present disclosure can generate a controlled level of heat sufficient to raise the temperature of the skin over which they are placed to about 36° C. to about 44° C. In one embodiment, the temperature modification apparatus can generate a controlled level of heat sufficient to raise the skin temperature of the skin over which the apparatus is placed to a temperature of about 39° C. to about 41° C. The temperature modification apparatus can achieve an increase in temperature to within one of the desired temperature ranges (above) within a 10 minutes following activation of the apparatus. In one embodiment, the temperature modification apparatus can achieve a temperature within the desired temperature range within about 5 minutes following activation of the apparatus. In another embodiment, the temperature modification apparatus can achieve a temperature within the desired temperature range within about 3 minutes following activation of the apparatus.

The temperature modification apparatuses can further be capable of maintaining the skin within the above temperature ranges for periods of time of about 1 minute to about 60 minutes. In one embodiment, the temperature modification apparatus can maintain the skin within the above temperature ranges for a period of time of about 1 minute to about 45 minutes. In another embodiment, the temperature modification apparatus can maintain the skin within the above temperature ranges for a period of time of about 3 minutes to about 35 minutes. In a further embodiment, the temperature modification apparatus can maintain the skin within the above temperature ranges for a period of time of about 5 minutes to about 30 minutes. Each of these periods or durations of controlled heating are examples of what is referred to herein as a “predetermined period of time.”

The temperature modification apparatuses disclosed herein can generate heat through a number of mechanisms. In one embodiment, the temperature modification apparatus can generate heat through chemical-based exothermic reactions. Other heating mechanisms can also be used, such as heating by phase transition of supersaturated solutions (such as phase transition of sodium acetate solutions), radiation (microwave or infrared, for example), electricity-resistor, combinations thereof, and/or other heating sources. In one embodiment, the temperature modification apparatus can include an infrared heating component. In another embodiment, the temperature modification apparatus can include a microwave heating component. In still a further embodiment, the temperature modification apparatus can include an electric heating component. Electric heating components can be powered by a variety of sources such as, for example, direct current (batteries) and/or alternating electric current. Electric heating components can be configured to provide a predetermined heating profile so that the heating profile is met automatically after engaging or turning on the electric device, e.g., use of timers, programmed electricity supply, finite battery power, etc. Alternatively, the heating profile can be met merely by providing heat at an appropriate temperature with an instruction to the user to remove the heating device after a specific period of time.

In one embodiment, the temperature modification apparatus can include a chemical heating component that generates heat by an exothermic oxidative chemical reaction. The chemical-based exothermic oxidation reaction can generate heat through the contact of the oxidative material or oxidizable component, e.g. iron or iron powder, with ambient air. U.S. Pat. No. 6,756,053, which is incorporated herein by reference in its entirety, describes examples of exothermic heating components and devices. The exothermic heating component can be provided in the form of an exothermic oxidation heating patch that is integrated with the transdermal nicotine patch or other system, or the heating patch can be a separate component that is placed over the transdermal nicotine patch or other system.

The amount of exothermic chemical composition in the temperature modification apparatus can vary depending on the desired duration of heating and the size of the temperature modification apparatus. It can be beneficial to limit the amount of the exothermic chemical composition in the heating component of the temperature modification apparatus, as a large amount of exothermic chemical composition can cause the heating component to be excessively large or cumbersome and impractical for use. In one aspect, the heating device can include from about 1 gram to about 3 grams of an exothermic chemical composition and can be configured to heat an area of skin ranging from about 8 cm² to 25 cm².

In addition to the oxidizable component (e.g. iron powder), the exothermic heating composition can further include activated carbon, salt (such as sodium chloride), and water. In one aspect, a water-retaining substance, such as vermiculite or wood powder, can also be included in the heating component. Thus, the oxidizable component, the activated carbon, salt, water, and other optional ingredients can be any of a number of specific compounds, such as those described in the previously mentioned U.S. Pat. No. 6,756,053. Depending on the configuration of the heating device, when stored for extended period of time, the exothermic heating components can generate gas (believed to be methane and hydrogen) which can cause the packaging, in which the exothermic heating component is present, to puff up which can cause complications and problems with respect to storage and transportation. To aid in combating these complications certain amounts of sulfur-containing compounds, or salts thereof, such as elemental sulfur, sulfates, sulfites, sulfides, or thiosulfates, can reduce or eliminate this gas generation problem when included in the packaging.

Water content in the exothermic chemical composition can have an impact on the heating temperature profile of the heating device. The weight ratio of water to the rest of the ingredients in the exothermic heating component can be in the range of about 1:1.5 to about 1:5. In another embodiment, the weight ratio of water to the rest of the ingredients in the exothermic heating component can be in the range of about 1:2.5 to about 1:4.5.

In one aspect, the exothermic chemical temperature modification apparatus can be manufactured in a manner so as to only have access to ambient oxygen through the holes in a cover that is made of air-impermeable material. In this way, the flow rate of oxygen from ambient air into the exothermic chemical composition is regulated, which in turn is a factor that can affect the amount and rate of heat generated by the heating component and the temperature of the skin surface on which the temperature control apparatus is applied. Other factors which can influence the temperature and heat generation of the heating component can be the size and surface area of the heating component, the amount of the exothermic chemical composition in the heating component, the number, size, and configuration of holes in the heating component's air impermeable cover material, the films and materials used in construction of the temperature modification apparatus, etc.

The temperature control apparatus disclosed herein can include an activation tab in order to facilitate on-demand activation and operation of the apparatus. The activation tab can be configured to be removed by the subject, on demand, typically after a pre-set period of time has passed following the application of the transdermal nicotine delivery system, e.g. after a steady state nicotine concentration is achieved. That being stated, activation of the apparatus can be effective to reduce nicotine cravings even before a steady state of nicotine in the blood is reached, as long as there is an accumulated depot of some degree beneath the skin surface. The ability of the subject to activate the temperature modification apparatus relates directly to the effectiveness of the disclosed apparatus and method, in that it allows the subject to treat their cravings or withdrawal systems on demand when the steady state or pre-activation nicotine plasma concentrations are insufficient.

The activation tab can be made of any air impermeable material that can be sealed or adhered over the holes of the temperature modification apparatus. In one embodiment, the activation tab can be sealed over the holes of the temperature modification apparatus by a heat seal. In another embodiment, the activation tab can be sealed over the holes of the temperature modification apparatus by an adhesive seal. In still another embodiment, the temperature modification apparatus can have more than one activation tab or the activation tab can be effectively configured to allow removal of more than one region of the tab while maintaining other portions of the tab intact over one or more of the holes in the temperature modification apparatus. In embodiments where the temperature control apparatus has more than one heating component, the apparatus may include multiple activation tabs, one for each of the heating components of the temperature modification apparatus. Temperature control apparatuses that have multiple heating components with individual activation tabs can be used to provide multiple episodes of heating over duration of application of a single transdermal nicotine delivery system.

The temperature modification apparatus of the present disclosure can be sufficiently large to cover all or a portion of the transdermal nicotine delivery system or the skin contact area of the transdermal nicotine delivery system. Thus, the size of the area heated by the temperature modification devices can have heating areas of about 2 cm² to about 100 cm², about 5 cm² to about 100 cm², about 7 cm² to about 75 cm², about 5 cm² to about 50 cm², or about 7 cm² to about 30 cm². In various other embodiments, other size ranges may be appropriate. In one embodiment, the temperature modification apparatus can have an area that is 20%, 15%, 10%, or 5% larger than the skin contact area of the transdermal nicotine delivery system. In such embodiments the temperature modification apparatus can have an adhesive around at least its perimeter and the adhesive can be used to adhere the temperature modification device onto the skin and/or transdermal nicotine delivery system for a period of at least 8 hours, at least 12 hours, or at least 24 hours. Any adhesive that is skin friendly and breathable can be used. Non-limiting examples can include acrylic adhesives, polyurethane adhesives, and other known skin friendly adhesives. Typically the temperature modification apparatus can have a thickness of about 10 mm or less. In one embodiment, the temperature modification apparatus can have a thickness of about 5 mm or less.

The temperature modification apparatus can have a size (area) that is such that more than one heating component can be applied to the transdermal nicotine system. In such an embodiment, each of the individual heating components can be configured to be individually activated, as mentioned above. Such a configuration can allow for repeated heating opportunities for the user over the wearing period of the transdermal nicotine delivery system. This can be particularly beneficial if the subject experiences multiple high intensity nicotine cravings during the wearing of a single transdermal nicotine delivery system. For example, the temperature modification apparatus can be re-activated a second time by activating a second heating element in the same apparatus after the steady state plasma concentration has been re-established. Alternatively, the temperature modification apparatus can be configured for a single use, but if the system is modular, the temperature modification apparatus can be removed and replaced with a second temperature modification apparatus in preparation for a second activating step.

The temperature modification apparatus and the transdermal nicotine delivery system can be manufactured, distributed, and even sold separately or they can be incorporated into a single integrated system. Thus, for example, in one embodiment, a kit of the present disclosure can be provided that includes a transdermal nicotine delivery system, (e.g. a nicotine patch, peel, plaster, or the like) and a separate temperature modification apparatus. The temperature modification apparatus can be configured to be applied directly to the skin following removal of the transdermal delivery system or it can be applied over or on top of the transdermal delivery system (e.g. to the external surface of the transdermal delivery system).

When the transdermal nicotine delivery system and the temperature modification apparatus are incorporated into a single system or device, the system can take on various configurations. In one embodiment, the transdermal nicotine delivery component of the integrated system can be a transdermal nicotine patch and the temperature modification apparatus can be an exothermic oxidation heating patch. When the two components are integrated into a single system, the integrated system (e.g. integrated patch) can have an area similar to the areas for the areas for the individual components of the systems described above, e.g. 5 cm² to about 100 cm² Whether provided and administered as separate individual components or as an integrated system, the temperature modification apparatus, the transdermal nicotine delivery system, or the integrated system can be manufactured in the form or substantially in the form of a variety of geometric shapes. For example, the components of the integrated system can be substantially oval, round, square, triangular, or rectangular in shape. The integration of the two components can be accomplished in a variety of ways including mechanical attachment via heat seal or adhesive seal or by some other means. The temperature modification apparatus may be smaller than the transdermal nicotine delivery system, the same size as the transdermal nicotine delivery system, or larger than the transdermal nicotine delivery component.

The methods and associated systems of the present disclosure are advantageous in that they can provide rapid increases in nicotine plasma concentration in a subject so as to provide nicotine levels that are elevated as compared to a steady state or pre-activation nicotine concentration achieved by a transdermal nicotine delivery system alone. This is particularly beneficial when a subject is experiencing nicotine cravings for which the steady state or pre-activation nicotine plasma concentration alone is not sufficient to satisfy. The increase in nicotine plasma concentrations following activation of the temperature modification apparatus is due to an increased rate of nicotine entering systemic circulation from the nicotine depot formed in the skin or sub-skin tissues following initial administration of the transdermal nicotine delivery system. For example, in one embodiment, the method of the present disclosure can, following activation of the temperature modification apparatus, provide an increase in a subject's nicotine plasma concentration from 5% to 50% as compared to the subject's steady state nicotine plasma concentration after application of a transdermal nicotine delivery system. In another embodiment, following activation of the temperature modification apparatus, the method can provide an increase a subject's nicotine plasma concentration of 15% to 40% as compared to the subject's steady state nicotine plasma concentration. In another embodiment, following activation of the temperature modification apparatus, the method can provide an increase a subject's nicotine plasma concentration of 20% to 35% as compared to the subject's steady state nicotine plasma concentration. In a further embodiment, following activation of the temperature modification apparatus, the method can provide an increase a subject's nicotine plasma concentration of 30% to 35% as compared to the subject's steady state nicotine plasma concentration. In still a further embodiment, the peak increase in the subject's plasma concentration (e.g. to 30% to 35%) can be achieved within about 10 to about 20 minutes following activation of the temperature modification apparatus.

Activation of the temperature modification apparatus results in an increase in the nicotine plasma concentration in the subject above the steady state level provided by the transdermal nicotine delivery system alone. In one embodiment, activation of the temperature modification apparatus can result in a nicotine Cₘₐₓ of about 6.0 ng/mL to about 8.5 ng/mL following a steady state or pre-activation concentration of about 5.5 ng/mL. In another embodiment, activation of the temperature modification apparatus can result in a nicotine Cₘₐₓof about 6.5 ng/mL to about 8.0 ng/mL following a steady state or pre-activation concentration of about 5.5 ng/mL. In a another embodiment, activation of the temperature modification apparatus can result in a nicotine Cₘₐₓof about 13 ng/mL to about 15.5 ng/mL a pre-activation or steady state concentration of about 10.5 ng/mL. In another embodiment, activation of the temperature modification apparatus can result in a nicotine Cₘₐₓof about 18 ng/mL to about 22.5 ng/mL can be achieved in the subject following a pre-activation or steady state concentration of about 16 ng/mL. In another embodiment, activation of the temperature modification apparatus can result in a nicotine Cₘₐₓof about 24 ng/mL to about 26.5 ng/mL can be achieved in the subject following a pre-activation or steady state concentration of about 19 ng/mL. In another embodiment, activation of the temperature modification apparatus can result in a nicotine Cₘₐₓof about 28 μg/L to about 32 μg/L can be achieved in the subject following a pre-activation or steady state concentration of about 22.5 μg/L. In a further embodiment, activation of the temperature modification apparatus can result in a nicotine Cₘₐₓof about 18 μg/L to about 21.5 μg/L can be achieved in the subject following a pre-activation or steady state concentration of about 15 μg/L. In still a further embodiment, activation of the temperature modification apparatus can result in a nicotine Cₘₐₓof about 8.5 μg/L to about 11 μg/L can be achieved in the subject following a pre-activation or steady state concentration of about 7.5 μg/L.

The rate of increase in the nicotine plasma concentration following activation of the temperature modification apparatus can be such that it is similar to the rate of increase experienced by a subject following the smoking of a cigarette. More specifically, after the activating step, the increase in the nicotine plasma concentration over time can have a slope that approximates an increase in nicotine plasma concentration in the subject upon smoking a cigarette. In another example, the slope of the rate of increase over time of the nicotine plasma concentration following activation of the temperature modification apparatus can be within 30%, 25%, 20%, 15%, or 10% of the rate of increase of nicotine plasma concentration following the smoking of a cigarette having 0.7 mg to 1.4 mg nicotine based on 10 normal inhalation puffs over a period of about 5 minutes.

Activation of the temperature modification apparatus can provide an increase in nicotine plasma concentration having a relatively steep mean slope of increase over a finite period of time. In another example, following the activating step, the increase in the nicotine plasma concentration over time can have a mean slope of about 0.1 ng/mL/min over a period of about 15 minutes to about 0.3 ng/mL/min over a period of about 15 minutes. In still another example, following the activating step, the increase in the nicotine plasma concentration over time has a mean slope of about 0.12 ng/mL/min over a period of about 15 minutes to about 0.25 ng/mL/minute over a period of about 15 minutes.

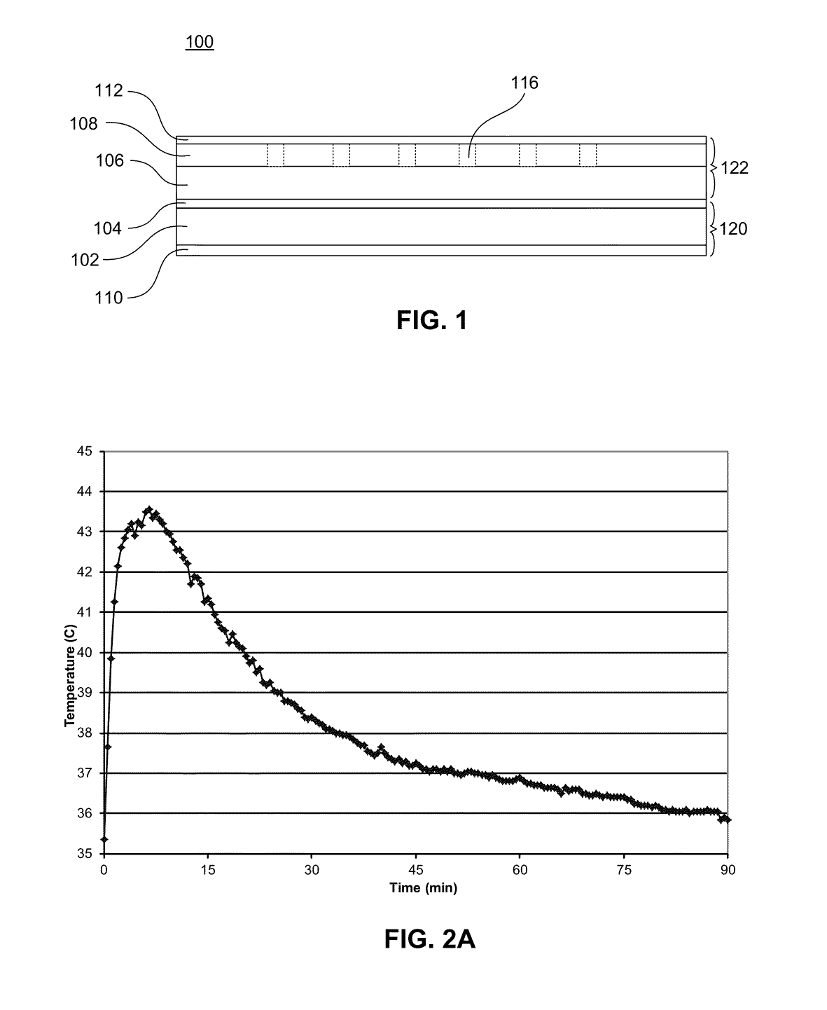

By way of example, FIG. 1 provides a schematic profile of one embodiment of an integrated system 100, including transdermal nicotine patch 120 and a temperature modification apparatus 122, which could be used in accordance with the methods of the present disclosure. The transdermal nicotine patch includes a drug layer 102, a backing layer 104, and a release liner 110. The temperature modification apparatus can include an exothermic chemical composition layer 106, an air-impermeable top cover film 108 having a plurality of holes 116 therein, and an air-impermeable activation tab 112 disposed on the upper surface thereof which inhibits air penetration through the plurality of holes until after removal of the activation tab. Once the activation tab is removed to expose the plurality of holes to ambient air, the holes allow for the passage of the ambient air through the air-impermeable top cover film to the exothermic chemical composition layer. In some embodiments, the layer of exothermic chemical composition can be disposed between the air-impermeable top cover film and the backing layer of the transdermal nicotine patch, as is shown. This backing layer can be moisture and air impermeable to prevent the active components of the temperature modification device and the active ingredients of transdermal nicotine delivery system from contacting one another. Additionally, a release liner 110 can be adhered to the lower surface of the transdermal nicotine delivery system for packing and storage, and can be removed prior to application on the application site of the subject. It is noted that though an integrated system is shown, the transdermal nicotine patch can be prepared as a standalone patch, and the temperature modification apparatus can also be prepared as a standalone device that is applied over the transdermal nicotine patch.

### EXAMPLES

The following example illustrates an embodiment of the disclosure that is presently best known. However, it is to be understood that the following is only exemplary or illustrative of the application of the principles of the present disclosure. Numerous modifications and alternative compositions, methods, and systems may be devised by those skilled in the art without departing from the spirit and scope of the present disclosure. The appended claims are intended to cover such modifications and arrangements. Thus, while the present disclosure has been described above with particularity, the following example provides further detail in connection with what are presently deemed to be the most practical and preferred embodiments of the disclosure.

### Example 1

### Assessment of Impact of Heat on the Systemic Delivery of Nicotine

A study was designed to analyze the effect of local heat administration in combination with transdermal nicotine. In particular, the study documented the effect of heat on the absorption of nicotine from currently marketed transdermal delivery systems.

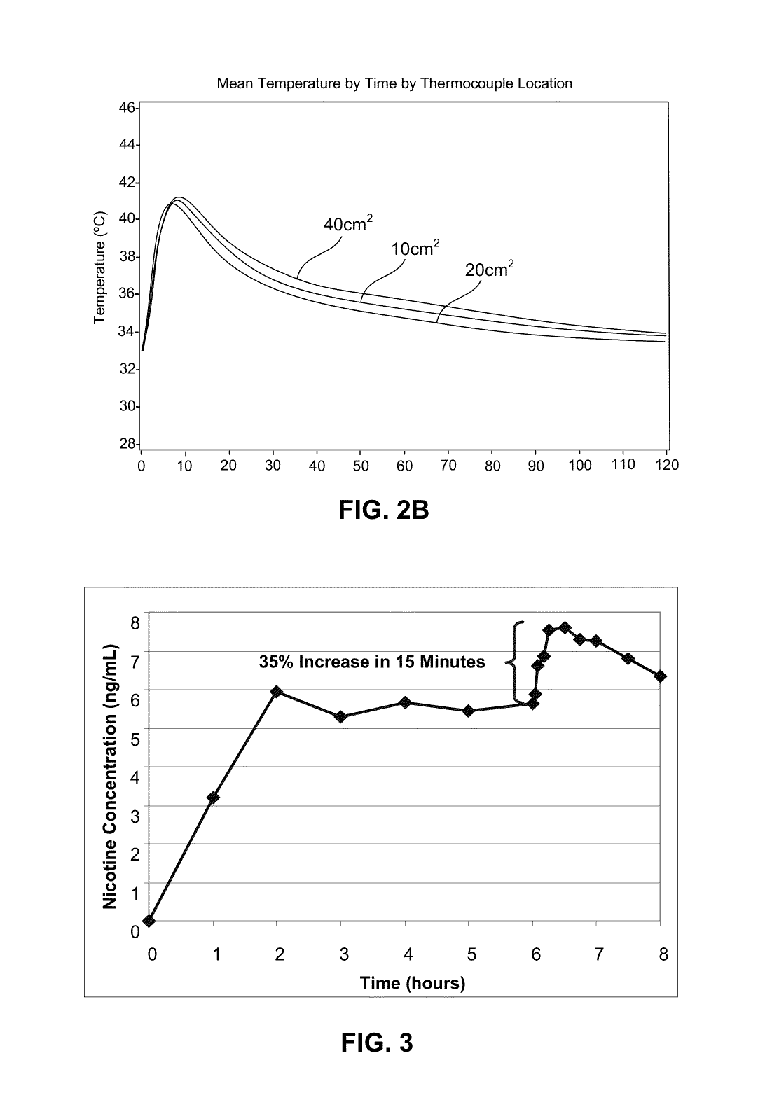

Twelve adult non-smoking, non-tobacco user volunteers were selected. A 7 mg Nicoderm® patch was applied to the arm of each of the subjects for a period of 24 hours. Six hours following the application of the Nicoderm® patch, an exothermic temperature modification device was placed on top of the Nicoderm® patch and activated. The exothermic temperature modification device produced heat sufficient to raise the average skin temperature of the skin over which it was applied to about 40° C. to 42° C. The temperature profile of the exothermic temperature modification apparatus is shown in FIG. 2. In this example, the exothermic temperature modification apparatus was maintained in place for a period of 15 minutes and then removed.

Blood samples were drawn hourly from each of the volunteers over the first 8 hours following application of the Nicoderm® patch. The mean nicotine concentrations for the 8 hour test period are shown in FIG. 3. As can be seen in FIG. 3, a steady state nicotine plasma concentration was achieved within about 2 hours following initial administration of the transdermal nicotine delivery system. Following application of the exothermic temperature modification apparatus, the mean nicotine plasma concentrations increased rapidly to levels that were about 30% to 35% higher than the steady state level that was achieved by application of the Nicoderm® patch alone. It is noteworthy that the increase in nicotine plasma concentrations following the application of the exothermic temperature modification device mimics the PK curves following smoking (but at lower concentrations) indicating that the use of the temperature modification apparatus in conjunction with a transdermal nicotine delivery system provides a benefit in helping treat breakthrough nicotine cravings and/or withdrawal symptoms.

It is noted that the above example is provided for exemplary purposes only. Thus, while the invention has been described with reference to certain embodiments, those skilled in the art will appreciate that various modifications, changes, omissions, and substitutions can be made without departing from the spirit of the disclosure. It is therefore intended that the disclosure be limited only by the scope of the appended claims.

What is claimed is:

1. A method for controlling the rate of nicotine delivery into systemic circulation of a subject, comprising:
transdermally administering nicotine at a topical administration site of a subject;
achieving a pre-activation or steady state plasma concentration of nicotine in the subject; and
activating a temperature modification apparatus over the topical administration site after achieving the steady state, wherein the temperature modification apparatus is configured to generate heat for a predetermined period of time; and
wherein about 5 to about 30 minutes following activation of the temperature modification apparatus the subject's nicotine plasma concentration increases by at least about 5% over the subject's steady state nicotine plasma concentration.

2. The method of claim 1, further comprising repeating the activating step after the nicotine plasma concentration returns to the steady state.

3. The method of claim 1, wherein the predetermined period of time is about 1 minute to about 45 minutes following activation.

4. The method of claim 1, wherein the predetermined period of time is about 3 minutes to about 30 minutes following activation.

5. The method of claim 1, wherein the predetermined period of time is about 5 minutes to about 25 minutes following activation.

6. The method of claim 1, wherein the nicotine plasma concentration in the subject following the activating step increases by about 10% to about 50% compared to the pre-activation or steady state nicotine plasma concentration.

7. The method of claim 1, wherein the nicotine plasma concentration in the subject following the activating step increases by about 15% to about 40% compared to the pre-activation or steady state nicotine plasma concentration.

8. The method of claim 1, wherein the nicotine plasma concentration in the subject following the activating step increases by about 20% to about 35% compared to the pre-activation or steady state nicotine plasma concentration.

9. The method of claim 1, wherein the nicotine plasma concentration in the subject following the activating step increases by 30% to 35% compared to the pre-activation or steady state nicotine plasma concentration.

10. The method of claim 1, wherein the nicotine plasma concentration in the subject following the activating step increases to its peak level at from about 10 to about 20 minutes following activation of the temperature modification apparatus.

11. The method of claim 10, wherein the peak level is an increase of about 30% to about 35%.

12. The method of claim 1, wherein following the activating step, the increase in the nicotine plasma concentration over time has a slope that approximates an increase in nicotine plasma concentration in the subject upon smoking a cigarette.

13. The method of claim 1, wherein following the activating step, the increase in the nicotine plasma concentration over time has a slope that is within about 30% of the rate of increase of nicotine plasma concentration following the smoking of a cigarette having 0.7 mg to 1.4 mg nicotine, wherein the smoking of the cigarette involves 10 normal inhalation puffs over a period of about 5 minutes.

14. The method of claim 1, wherein following the activating step, the increase in the nicotine plasma concentration over time has a mean slope of about 0.1 ng/mL/min over a period of about 15 minutes to about 0.3 ng/mL/min over a period of about 15 minutes.

15. The method of claim 1, wherein following the activating step, the increase in the nicotine plasma concentration over time has a mean slope of about 0.12 ng/mL/min over a period of about 15 minutes to about 0.25 ng/mL/minute over a period of about 15 minutes.

16. The method of claim 1, wherein following the activating step, a concentration (Cₘₐₓ) of nicotine of about 6.5 ng/mL to about 8 ng/mL is achieved in the subject following a pre-activation or steady state concentration of about 5.5 ng/mL.

17. The method of claim 1, wherein following the activating step, a nicotine Cₘₐₓof about 13 ng/mL to about 15.5 ng/mL is achieved in the subject following a pre-activation or steady state concentration of about 10.5 ng/mL.

18. The method of claim 1, wherein following the activating step, a nicotine Cₘₐₓof about 18 ng/mL to about 22.5 ng/mL is achieved in the subject following a pre-activation or steady state concentration of about 16 ng/mL.

19. The method of claim 1, wherein following the activating step, a nicotine Cₘₐₓof about 24 ng/mL to about 26.5 ng/mL is achieved in the subject following a pre-activation or steady state concentration of about 19 ng/mL.

20. The method of claim 1, wherein following the activating step, a nicotine Cₘₐₓof about 28 μg/L to about 32 μg/L is achieved in the subject following a pre-activation or steady state concentration of about 22.5 μg/L.

21. The method of claim 1, wherein following the activating step, a nicotine Cₘₐₓof about 18 μg/L to about 21.5 μg/L is achieved in the subject following a pre-activation or steady state concentration of about 15 μg/L.

22. The method of claim 1, wherein following the activating step, a nicotine Cₘₐₓof about 8.5 μg/L to about 11 μg/L is achieved in the subject following a pre-activation or steady state concentration of about 7.5 μg/L.

23. The method of claim 1, wherein the temperature modification apparatus generates a controlled level of heat between about 36° C. to about 44° C.

24. The method of claim 1, wherein the temperature modification apparatus generates a controlled level of heat between about 39° C. to about 41° C.

25. The method of claim 1, wherein temperature modification apparatus comprises an electric, infrared, chemical, or microwave heating component.

26. The method of claim 1, wherein temperature modification apparatus comprises a chemical heating component.

27. The method of claim 26, wherein the chemical heating component is an exothermic oxidation heating patch.

28. The method of claim 27, wherein nicotine is administered with a transdermal nicotine patch.

29. The method of claim 28, wherein the transdermal nicotine patch and the exothermic oxidation heating patch are configured as an integrated patch.

30. The method of claim 29, wherein the integrated patch is from about 5 cm² to about 100 cm².

31. The method of claim 29, wherein the integrated patch is substantially oval, round, square, triangular, or rectangular in shape.

32. The method of claim 28, wherein the transdermal nicotine patch and the exothermic oxidation heating patch are separate devices configured so that the exothermic oxidative heating patch is applied over the transdermal nicotine patch.

33. The method of claim 1, wherein the temperature modification apparatus is configured to be activated more than once.

34. The method of claim 33, further comprising the step of re-activating the temperature modification apparatus a second time after the activating step is completed and the steady state plasma concentration has been re-established.

35. The method of claim 1, wherein the temperature modification apparatus is configured for a single use, and the method further comprises removing the temperature modification apparatus after use and replacing it with a second temperature modification apparatus in preparation for a second activating step.

36. The method of claim 35, further comprising activating the second temperature modification apparatus over the topical administration site after re-establishing the steady state.

37. A temperature modification apparatus for use with a transdermal nicotine delivery system, said apparatus, comprising:
an exothermic chemical composition layer,
an air impermeable layer disposed on an upper surface of the chemical composition layer and having one or more holes therein,
an activation tab removably adhered to an upper surface of the air impermeable layer and being configured to cover the one or more holes in the air impermeable layer and inhibit the passage of air through the holes prior to removal of the activation tab, and
an adhesive layer disposed on a lower surface of one or both of the exothermic chemical composition layer and the lower surface of the air impermeable layer, said adhesive layer being configured to adhere the temperature modification apparatus to one or both of a skin surface and a transdermal nicotine delivery system.

38. The temperature modification apparatus of claim 37, wherein the temperature modification apparatus generates a controlled level of heat from about 36° C. to about 44° C.

39. The temperature modification apparatus of claim 37, wherein the temperature modification apparatus generates a controlled level of heat from about 39° C. to about 41° C.

40. The temperature modification apparatus of claim 37, wherein temperature modification apparatus is configured to be adhered to both the skin surface of the transdermal nicotine delivery system.

41. The temperature modification apparatus of claim 37, wherein temperature modification apparatus is configured to be adhered to the transdermal nicotine delivery system, but not the skin surface.

42. The temperature modification apparatus of claim 37, wherein the temperature modification apparatus has an area of about 5 cm² to about 100 cm².

43. The temperature modification apparatus of claim 37, wherein the integrated patch is substantially oval, round, square, triangular, or rectangular in shape.

44. The temperature modification apparatus of claim 37, wherein the activation tab is configured for removal by a subject wearing the temperature modification apparatus.

45. The temperature modification apparatus of claim 37, comprising multiple heating components, each comprising the exothermic chemical composition layer, the air impermeable layer having one or more holes therein, and the activation tab removably adhered to an upper surface of the air impermeable layer and being configured to cover the one or more holes in the air impermeable layer and inhibit the passage of air through the holes prior to removal of the activation tab, thereby providing individual activation tabs useable to provide multiple episodes of heating over the duration of application of a single transdermal nicotine delivery system.
